(19) United States
(12) Patent Application Publication
(10) Pub. No.: US 2023/0389551 A1
(43) Pub. Date: Dec. 7, 2023

(54) Premoistened Wipes with Virucidal Properties Against Non-Enveloped Viruses

(71) Applicant: ARXADA, LLC, Morristown, NJ (US)

(72) Inventors: Xiao Jiang, Montvale, NJ (US); David Jones, Allendale, NJ (US); Andrew Kloeppel, Harrington Park, NJ (US); Andrew Kaziska, Whitehouse Station, NJ (US); Craig Carter, Macungie, PA (US)

(21) Appl. No.: 18/453,429

(22) Filed: Aug. 22, 2023

Related U.S. Application Data
(62) Division of application No. 16/104,607, filed on Aug. 17, 2018, now abandoned.
(60) Provisional application No. 62/547,219, filed on Aug. 18, 2017.

(51) Int. Cl. A01N 47/44; A01N 25/34; A01N 33/04; A01N 33/12

(52) U.S. Cl. CPC A01N 47/44; A01N 25/34; A01N 33/04; A01N 33/12

(57) ABSTRACT

A presaturated wiping product is disclosed well suited for destroying non-enveloped viruses, including the Norovirus. The wiping composition contains a first anti-microbial agent blended with a second agent, such as a preservative. Even at very low concentrations within the wiping composition, the wiping products can completely destroy and kill non-enveloped viruses at relatively short contact times.

### RELATED APPLICATIONS

The present application is based upon and claims priority to U.S. Provisional Application Ser. No. 62/547,219, filed on Aug. 18, 2017, which is incorporated herein by reference.

### BACKGROUND

Pathogenic organisms, such as bacteria, fungi, and viruses, continue to cause infections in humans as well as domestic animals and pets. Disinfectant formulations have been developed over the last several decades to reduce or destroy pathogenic organisms and accordingly, reduce the rate of infection. Literally any hard surface including floors, walls, countertops, windows, windowsills, sinks, faucets, waste containers, appliances, and cabinet surfaces can become contaminated. Disinfectants have been developed to treat hard surfaces for use in hospitals, rest homes, schools, and homes. Anti-microbial compositions for decontamination, disinfection and/or sanitization can be added to wipes in order to treat such surfaces.

One particular class of anti-microbial compositions includes quaternary ammonium compounds, also known as “quats”, as a microbial control agent. The use of quats as biocides is well known. Certain amine biocides have also been used in the past.

In one application, the anti-microbial composition may be incorporated into a wiping product and packaged as a premoistened wipe. These products are generally inexpensive to manufacture, are easy to use, and are disposable after use. Premoistened wipes impregnated with a quat-based anti-microbial composition have been found to provide a broad range of activity against many different types of microorganisms including many different strains of bacteria and have also shown to demonstrate very short kill times. Quat-based formulations and other biocide formulations incorporated into premoistened wipes, however, have demonstrated in some applications a lack of effectiveness against certain viruses.

Viruses are divided into families on the basis of size, symmetry, type of nucleic acid genome or deoxyribonucleic acid and mode of replication. Viruses of different families, for instance, vary in their resistance to disinfectants. For instance, enveloped viruses are usually more sensitive to disinfectants and thus are more easily controlled than non-enveloped viruses. Non-enveloped viruses have a complex outer structure and are more resistant to not only disinfectants, but also harsh environmental conditions. One example of a non-enveloped virus is the Norovirus. The Norovirus can cause viral gastroenteritis in humans. The Norovirus can affect people of all ages. Outbreaks of the Norovirus infection can occur, for instance, in closed or semi-closed communities, such as long-term care facilities, overnight camps, hospitals, schools, prisons, clubs, dormitories, and cruise ships, where the infection can spread very rapidly.

In view of the above, a need currently exists for a disinfectant composition that has a broader range of anti-microbial properties, especially against viruses such as non-enveloped viruses. A need also exists for a method for enhancing the antimicrobial properties of a premoistened wipe in a manner that makes the premoistened wipe effective against non-enveloped viruses such as the Norovirus.

### SUMMARY

In general, the present disclosure is directed to a wiping composition having anti-microbial properties. The wiping composition contains a blend of anti-microbial and/or preservative agents that provides the wiping composition with a broad range of activity against many different microorganisms, including viruses such as non-enveloped viruses. In addition to two or more anti-microbial and/or preservative agents, the wiping composition can contain various other components such as organic solvents, an alkanolamine, a chelating agent, one or more surfactants, and/or a liquid carrier. The wiping composition is well suited for disinfecting hard surfaces. Of particular advantage, the wiping composition of the present disclosure not only demonstrates quick disinfecting performance against many hospital grade microorganisms such as Staphylococcus aureus and Pseudomonas aeruginosa but can also destroy and kill disinfectant-resistant viruses, such as the Norovirus.

In one embodiment, for instance, the wiping composition of the present disclosure comprises an aqueous solution containing a first anti-microbial agent blended with a preservative. The first anti-microbial agent, for instance, may comprise a quaternary ammonium cation or a tertiary amine. The preservative, on the other hand, can comprise a polyhexamethylene biguanide or salt thereof. The quaternary ammonium cation may comprise a halide salt of a quaternary ammonium cation. For instance, the quaternary ammonium cation may comprise an alkyl dimethyl benzyl ammonium chloride, a dialkyl dimethyl ammonium chloride, an alkyl dimethyl ethyl benzyl ammonium chloride, or mixtures thereof. Alternatively, the quaternary ammonium cation may comprise a carbonate/bicarbonate salt of a quaternary ammonium cation.

The first anti-microbial agent and the preservative can be present within the wiping composition at a concentration sufficient to completely inactivate various viruses when tested according to AOAC Test Method 961.02 and ASTM Test E2362 for Presaturated Towelettes after a contact time of 10 minutes or 5 minutes. For instance, the virus completely inactivated may comprise Feline Calicivirus or Norovirus.

In one embodiment, the first anti-microbial agent can be present in the wiping composition in relation to the preservative or second agent such that the weight ratio between the first anti-microbial agent and the preservative is from about 50:1 to about 1:10, such as from about 20:1 to about 1:8, such as from about 15:1 to about 1:5, such as from about 10:1 to about 1:3. In one embodiment, the wiping composition may comprise a concentrate that is intended to be diluted prior to use. For instance, the concentrate can contain water in an amount from about 10% to about 65% by weight. Once diluted, the wiping composition can contain water in an amount greater than about 95% by weight, such as in an amount greater than about 96% by weight, such as in an amount greater than about 98% by weight. In one embodiment, the concentrate is formulated such that one part of concentrate is added to from about 100 parts to about 300 parts of water. The wiping composition can have a pH of 8 or greater, such as 8.5 or greater, such as 9 or greater, such as 9.5 or greater, such as 10 or greater.

Various other ingredients and components can be contained in the wiping composition. For instance, the wiping composition can contain a pH builder, one or more nonionic surfactants, a chelating agent, and/or an organic solvent.

In one embodiment, a surfactant is present comprising an ethoxylated alcohol. The ethoxylated alcohol, for instance, may comprise an ethoxylated C₁₂ to C₁₄ alkyl alcohol. Alternatively, the surfactant may comprise a glycoside and/or a C₁₂-C₁₅ secondary alkanol with ethylene oxide units, such as an alkyloxypolyethylene oxyethanol. Each surfactant can generally be present in the composition in an amount from about 0.01% to about 2% by weight.

The wiping composition may further contain a chelating agent. The chelating agent, for instance, may comprise tetrasodium EDTA. The chelating agent can generally be present in the composition in an amount from about 0.01% to about 2% by weight.

The wiping composition may further contain a pH builder. The pH builder, for instance, may be added in order to increase the pH of the composition. pH builders that may be used in accordance with the present disclosure include alkanolamines such as monoethanolamine, hydroxides such as alkali or alkaline earth metal hydroxides, carbonates, bicarbonates, borates, silicates such as metasilicate, or mixtures thereof.

In one embodiment, the present disclosure is directed to a premoistened wiping product containing the wiping composition described above. The premoistened wiping product may comprise a liquid absorbent substrate impregnated with the wiping composition. The liquid absorbent substrate may comprise a meltblown web, a coform web, a spunbond web, an airlaid web, an airlaced web, a hydroentangled web, a bonded carded web, or a laminate thereof.

As described above, the wiping composition incorporated into the premoistened wiping product contains a first anti-microbial agent combined with a second agent, such as a preservative. The first anti-microbial agent can comprise a quaternary ammonium cation and/or a tertiary amine and can be present in the wiping composition in an amount less than about 2% by weight, such as in an amount less than about 1.5% by weight, such as in an amount less than about 1% by weight, such as in an amount less than about 0.5% by weight, such as in an amount less than about 0.4% by weight. In fact, in one embodiment, the first anti-microbial agent can be present in the wiping composition in an amount less than 0.1% by weight. Similarly, the second agent or preservative comprising a polyhexamethylene biguanide or salt thereof can be present in the wiping composition in an amount less than about 1.5% by weight, such as in an amount less than about 1% by weight, such as in an amount less than about 0.5% by weight, such as in an amount less than about 0.3% by weight, such as in an amount less than about 0.1% by weight. For instance, the polyhexamethylene biguanide or salt thereof can be present in the wiping composition in an amount less than about 1,000 ppm, such as in an amount less than about 800 ppm, such as in an amount less than about 700 ppm, such as in an amount less than about 600 ppm, such as in an amount less than about 500 ppm. The polyhexamethylene biguanide or salt thereof is generally present in the wiping composition in an amount greater than about 10 ppm.

In one particular embodiment, the wiping composition contains a quaternary ammonium chloride in combination with a metasilicate and/or a monoethanolamine, a chelating agent, and a nonionic surfactant comprising a C₁₁-C₁₅ alkyl polyethyleneoxyethanol. In an alternative embodiment, the wiping composition can contain a quaternary ammonium carbonate/bicarbonate salt, a glycoside surfactant and an organic solvent, such as isopropanol in combination with the polyhexamethylene biguanide or salt thereof.

The present disclosure is also directed to a method for increasing the effectiveness of a premoistened wipe against various microorganisms, such as non-enveloped viruses. The method includes the step of adding to a premoistened wipe a composition containing a first antimicrobial agent as described above with a preservative comprising a biguanide compound. The first antimicrobial agent and the biguanide compound are incorporated into the premoistened wipe at a concentration sufficient to control and destroy non-enveloped viruses that come in contact with the wipe, such as the norovirus.

Other features and aspects of the present disclosure are discussed in greater detail below.

### DETAILED DESCRIPTION

It is to be understood by one of ordinary skill in the art that the present discussion is a description of exemplary embodiments only, and is not intended as limiting the broader aspects of the present disclosure.

The present disclosure is generally directed to an anti-microbial wiping composition well suited for saturating wipes and using the wipes to disinfect various surfaces. The present disclosure is also directed to a method for increasing the effectiveness of a presaturated wipe against viruses, including non-enveloped viruses. In accordance with the present disclosure, the wiping composition contains a first anti-microbial agent blended with a preservative or second agent in a manner such that the anti-microbial agent synergistically works together with the preservative to destroy and kill many different types of microorganisms including viruses. Many disinfectant compositions, for instance, are ineffective against non-enveloped viruses that have a complex outer structure and are more resistant to harsh environmental conditions. These viruses can be particularly dangerous. For instance, the Norovirus, which is a non-enveloped virus, can cause viral gastroenteritis in humans and can quickly multiple when not controlled leading to outbreaks in closed or semi-closed communities, such as long-term care facilities, overnight camps, hospitals, schools, prisons, clubs, dormitories, cruise ships, and the like. Of particular advantage, the wiping composition of the present disclosure due to the blend of anti-microbial agent and preservative is capable of controlling and killing the Norovirus.

For example, a premoistened wipe saturated with the wiping composition of the present disclosure is capable of completely inactivating the Norovirus and other similar microorganisms, such as Feline Calicivirus, when tested according to AOAC Test Method 961.02 and ASTM Test E2362 for Presaturated Towelettes at contact times of 10 minutes or less, such as 8 minutes or less, such as 6 minutes or less, such as 5 minutes or less, such as 4 minutes or less, such as even 3 minutes or less.

In general, the wiping composition of the present disclosure contains a quaternary ammonium cation and/or a tertiary amine combined with a second agent or preservative, which may comprise a biguanide compound. The use of the two different agents not only enhances the spectrum of activity of the wiping composition against multiple microorganisms, but also is very effective against many viruses, including non-enveloped viruses. It was also unexpectedly discovered that efficacy against non-enveloped viruses can occur at extremely low concentrations.

In one embodiment, the first anti-microbial agent may comprise a quaternary ammonium carbonate. A quaternary ammonium carbonate can be represented by the following formula:

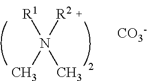

wherein R¹ is a C₁-C₂₀ alkyl or aryl-substituted alkyl group and R² is a C₈-C₂₀ alkyl group, and preferably wherein R¹ is the same as R² and R¹ is a C₈-C₁₂ alkyl group, as well as compositions further comprising the corresponding quaternary ammonium bicarbonate

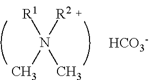

wherein R¹ is the same or a different C₁-C₂₀ alkyl or aryl-substituted alkyl group as above and R² is the same or a different C₈-C₂₀ alkyl group as above, but preferably wherein R¹ is the same as R² and R¹ is a C₈-C₁₂ alkyl group.

In one embodiment, the first anti-microbial agent contained in the composition comprises a di C₈-C₁₂ alkyl ammonium carbonate/bicarbonate. For example, in one particular embodiment, the anti-microbial or preservative composition contains didecyl dimethyl ammonium carbonate and didecyl dimethyl ammonium bicarbonate.

In other embodiments, however, the carbonate/bicarbonate salts of quaternary ammonium cations may be selected from dioctyldimethylammonium carbonate, decyloctyldimethylammonium carbonate, benzalkonium carbonate, benzethonium carbonate, stearalkonium carbonate, cetrimonium carbonate, behentrimonium carbonate, dioctyldimethylammonium bicarbonate, decyloctyldimethylammonium bicarbonate, benzalkonium bicarbonate, benzethonium bicarbonate, stearalkonium bicarbonate, cetrimonium bicarbonate, behentrimonium bicarbonate, and mixtures of one or more such carbonate salts.

In another embodiment, the first anti-microbial agent may comprise a quaternary ammonium halide. The quaternary ammonium halide may comprise, for instance, an alkyl quaternary ammonium halide or a benzyl ammonium halide.

Quaternary ammonium compounds, also known as “quats”, typically comprise at least one quaternary ammonium cation with an appropriate anion. Quats will generally have the general formula (1).

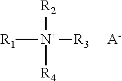

The groups R₁, R₂, R₃ and R₄ can vary within wide limits and examples of quaternary ammonium compounds that have anti-microbial properties will be well known to the person of ordinary skill in the art. Typically, two of R₁, R₂, R₃ and R₄ are lower alkyl, meaning having 1 to 4 carbon atoms, such as methyl, ethyl, propyl or butyl groups. In addition, two of R₁, R₂, R₃ and R₄ are longer chain alkyl groups of 6 to 24 carbon atoms, or a benzyl group. A⁻ is a monovalent anion or one equivalent of a polyvalent anion of an inorganic or organic acid. Suitable anions for A⁻ are in principle all inorganic or organic anions, in particular halides, for example chloride or bromide, carboxylates, sulfonates, phosphates or a mixture thereof.

In one embodiment, the quaternary ammonium compound may have the following R groups: R₁ is benzyl or C₆₋₁₈-alkyl, R₂ is C₁₋₁₈-alkyl or —[(CH₂)₂—O]ₙR₅ where n=1-20, R₃ and R₄ independently of one another are C₁₋₄-alkyl, R₅ is hydrogen or unsubstituted or substituted phenyl, and A⁻ is a monovalent anion or one equivalent of a polyvalent anion of an inorganic or organic acid.

In one embodiment, the quaternary ammonium compound may comprise a dialkyl ammonium compound, such as a dimethyl dialkyl ammonium compound. In one embodiment, the dimethyl dialkyl ammonium compound may have between about 8 and about 12 carbon atoms, such as from about 8 to about 10 carbon atoms in each of the alkyl groups.

Examples of dimethyl dialkyl ammonium compounds which may be used as the first anti-microbial agent include dimethyl dioctyl ammonium compounds such as dimethyl dioctyl ammonium chloride, dimethyl didecyl ammonium compounds such as dimethyl didecyl ammonium chloride and the like. Mixtures of dimethyl dialkyl ammonium compounds may also be used, and other anions, such as those described above, may also be used.

In an alternative embodiment, the first anti-microbial agent may comprise a benzyl ammonium compound, such as an alkyl dimethyl benzyl ammonium compound. In general, the alkyl group may contain from about 10 to about 18 carbon atoms, such as from about 12 to about 16 carbon atoms.

Examples of alkyl dimethyl benzyl ammonium compounds useable as the first anti-microbial agent include C₁₂ alkyl dimethyl benzyl ammonium chloride, C₁₄alkyl dimethyl benzyl ammonium chloride, and C₁₆ alkyl dimethyl benzyl ammonium chloride. In addition, a mixture of these alkyl dimethyl benzyl ammonium compounds can be used. For instance, the alkyl dimethyl benzyl ammonium compounds may include blends of C₁₂, C₁₄, and C₁₆alkyl dimethyl benzyl ammonium chlorides. Generally, it is preferable that the alkyl dimethyl benzyl ammonium compound, when a blend, contains higher concentrations of C₁₂ alkyl and C₁₄ alkyl components than C₁₆ alkyl components. It is noted that other anions, including those mentioned above may also be used.

In still another embodiment, the first anti-microbial agent may comprise a quaternary ammonium propionate. The quaternary ammonium propionate, for instance, may comprise a poly(oxyalkyl)ammonium propionate. In one particular embodiment, for instance, the first anti-microbial agent may comprise N,N-didecyl-N-methyl-poly(oxyethyl)ammonium propionate.

In addition to quaternary ammonium cations, the first antimicrobial agent may comprise a amine. The amine can be present alone or In combination with a quaternary ammonium cation.

Suitable amines include, but are not limited to, tertiary amines, such as (C6-C16) alkyl amines. The term “(C6-C16) alkyl amine” encompasses all amines which contain a (C6-C16) alkyl group. One (C6-C16) alkyl amine is N,N-bis(3-aminopropyl)dodecylamine, available as Lonzabac® 12.30 and 12.100 from Lonza, Inc. The chemical structure for N, N-Bis (3-aminopropyl) dodecylamine is as follows:

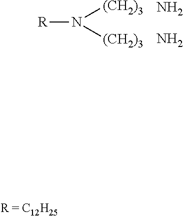

Other exemplary tertiary amines include, for example, N-(3-aminopropyl)-N-dodecyl propane-1,3-diamine, N-(3-aminopropyl)-N-decyl-1,3-propanediamine, N-(3-aminopropyl)-N-tetradecyl-1,3-propanediamine as well as their acid addition compounds. Other similar tertiary amines may be used.

The first antimicrobial agent as described above is combined with at least a second agent, such as a preservative, in accordance with the present disclosure. The second agent is selected that synergistically operates with the first anti-microbial agent to destroy and/or inhibit the growth of a target microorganism or a plurality of target microorganisms including non-enveloped viruses such as the Norovirus. Thus, the second agent or preservative acts as an adjuvant for the first anti-microbial agent.

In one embodiment, the preservative may comprise a guanidine, and particularly a biguanide and/or its substitution products, salts, analogs, derivatives, and/or combinations thereof. Biguanide is commonly represented by the following formula, though it is known to exist in other forms.

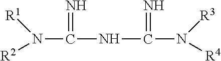

wherein R¹, R², R³ and R⁴ are each independently chosen from hydrogen, optionally substituted alkyl, optionally substituted phenyl, ethylene glycol, diethylene glycol, methylene glycol and tetraethylene glycol, or one of R¹, R². R³ and R⁴ may be

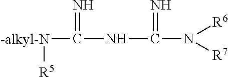

where R⁵, R⁶ and R⁷ are each independently chosen from hydrogen, optionally substituted alkyl, optionally substituted phenyl, ethylene glycol, diethylene glycol, methylene glycol and tetraethylene glycol. Substituents for the alkyl and phenyl groups include but are not limited to halo, e.g. chloro, bromo, fluoro or iodo, hydroxy and amino. The alkyl groups may have from 1 to 6 carbons, and may be saturated or unsaturated, straight chain or branched.

In one embodiment, the preservative may comprise a polymeric biguanide, otherwise known as a polybiguanide, or a salt, analog, or derivative thereof. In one embodiment, the polybiguanide may be a copolymer or a heteropolymer. The polybiguanide may be linear, branched, circular, and/or dendrimeric. The number of polymer repeating units can vary from 2 to 1,000, such as from 5 to 750, such as from 10 to 500, such as from 25 to 250, such as from 50 to 100 repeating units. In one specific embodiment, the polybiguanide may comprise polyhexamethylene biguanide (PHMB), polyhexamethylene monoguanide (PHMG), polyethylene biguanide (PEB), polytetramethylene biguanide (PTMB), polyethylene hexamethylene biguanide (PHMB), polymethylene biguanides (PMBs), poly(allylbiguanidnio-co-allyamine, poly(N-vinyl-biguanide), polyallylbiguanide etc.

For example, in one particular embodiment, the preservative may comprise a polyalkylene biguanide, such as polyhexamethylene biguanide. In one embodiment, the preservative may comprise polyhexamethylene biguanide hydrochloride (PHMB), also known as polyaminopropyl biguanide (PABP).

PHMB is commonly represented by the following formula, though it is known to exist as a complex mixture of polymeric biguanides with various terminal groups including guanidine (not shown).

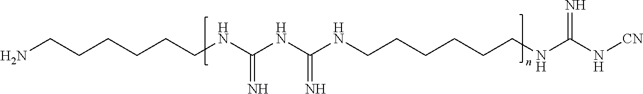

The value n represents the number of repeating units of the biguanide polymer.

More particularly, PHMB can be a mixture of various biguanide polymers that can include different combinations of terminal groups, e.g., amine, cyanoguanidino, and guanidine. Based only on these three terminal groups, at least six possible biguanide polymers can exist. There can be one biguanide polymer with two terminal amine groups, which is referred to as PHMB-AA, one with two terminal cyanoguanidino groups, which is referred to as PHMB-CGCG, and one with two terminal guanidine groups, which is referred to as PHMB-GG (see, below). There are also the three possible biguanide polymers having a combination of two different terminal groups. Again, based on the above terminal groups they include amine-cyanoguanidino (PHMB-ACG), amine-guanidino (PHMB-AG) and guanidine-cyanoguanidino (GCG). Accordingly, a sample of PHMB may comprise a mixture of polymeric biguanides with the three mentioned terminal groups. Moreover, some of the composition can include in-chain polymeric guanide (not shown). The subscript “n” represents the average number of repeating groups, and a distribution of polymer length exists for each of the polymers shown below.

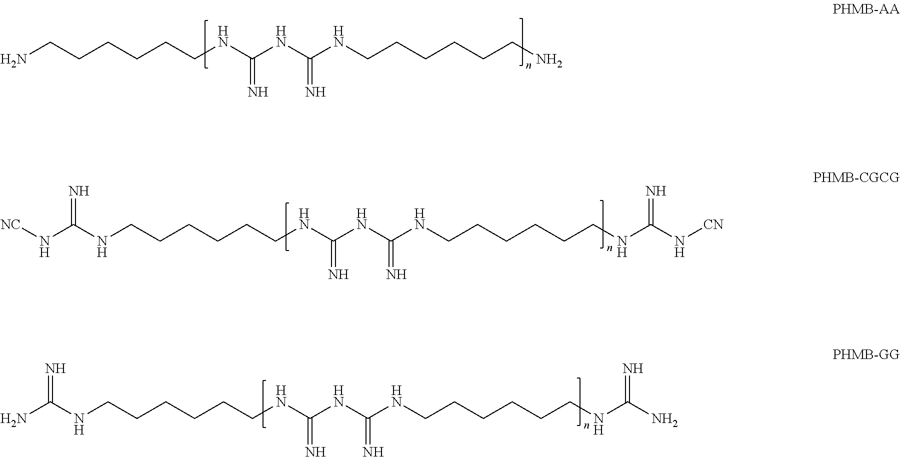

wherein n can be from about 1 to about 50, such as from about 1 to about 20.

Polyhexamethylene biguanide, such as polyhexamethylene biguanide hydrochloride, has a broad anti-microbial range and is fast acting. Further, the anti-microbial agent is stable over a broad pH range.

In one embodiment, the preservative may comprise a bis-biguanide. Bis-biguanide is commonly represented by the following formula, though it is known to exist in other forms.

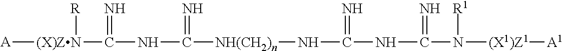

wherein A and A¹ each represent either (1) a phenyl radical which optionally is substituted by an alkyl or alkoxy group containing from 1 to about 4 carbon atoms, a nitro group, or a halogen atom; (2) an alkyl group containing from 1 to about 12 carbon atoms; or (3) alicyclic groups containing from 4 to about 12 carbon atoms; wherein X and X¹ each represent an alkylene radical containing from 1 to 3 carbon atoms; wherein Z and Z¹ each can be either 0 or 1; wherein R and R¹ each represent either hydrogen, or alkyl radical containing from 1 to about 12 carbon atoms, or an aralkyl radical containing from 7 to about 12 carbon atoms; wherein n is an integer from 2 to 12 inclusive; and wherein the chain (CH₂)ₙ may optionally be interrupted by oxygen or sulfur atoms, aromatic nuclei, etc. or substituted with halide, hydroxyl, alkyl, alkenyl, alkynl, or acetyl groups, aromatic nuclei, etc. In one embodiment, the chain (CH₂)ₙ may optionally be replaced by a bivalent bridging group, wherein the bivalent bridging group may be chosen from but is not limited to alkylenes, alicyclic groups, cyclic nuclei, aromatic nuclei etc. which may be substituted with or interrupted by oxygen or sulfur atoms, aromatic nuclei, etc. Exemplary bis-biguanide compounds include but are not limited to chlorhexidine, alexidine, trifluoromethyl phenyl bis-biguanide, analogs, derivatives, and/or salts thereof.

When the wiping composition is in a ready to use form and is saturated into a wiper, the first anti-microbial agent and the preservative may be contained in the wiping composition at relatively minor amounts and still have effectiveness against a broad range of microorganisms including non-enveloped viruses. For instance, one or more quaternary ammonium compounds and/or amine compounds can be present in the composition in an amount less than about 2% by weight, such as in an amount less than about 1.5% by weight, such as in an amount less than about 1% by weight, such as in an amount less than about 0.6% by weight, such as in an amount less than about 0.4% by weight, such as in an amount less than about 0.3% by weight, such as in an amount less than about 0.2% by weight. In one embodiment, for instance, the quaternary ammonium compound and/or amine compound can be present in the wiping composition in an amount less than 0.1% by weight, such as in an amount less than about 0.8% by weight, such as in an amount less than about 0.6% by weight. One or more quaternary ammonium compounds, for instance, can be present in the composition in an amount greater than about 0.001% by weight.

Similarly, the preservative or biguanide compound can be present in the wiping composition in an amount generally less than about 2% by weight, such as in an amount less than about 1.8% by weight, such as in an amount less than about 1.6% by weight, such as in an amount less than about 1.4% by weight, such as in an amount less than about 1.2% by weight, such as in an amount less than about 1% by weight, such as in an amount less than about 0.8% by weight, such as in an amount less than about 0.6% by weight, such as in an amount less than about 0.4% by weight, such as in an amount less than about 0.2% by weight, such as even in amounts less than 0.1% by weight. For instance, one or more biguanide compounds can be present in the wiping composition in an amount less than about 0.08% by weight, such as in an amount less than about 0.06% by weight. For example, one or more biguanide compounds can be present in the composition at a concentration of less than about 800 ppm, such as less than about 600 ppm, such as less than about 500 ppm, such as less than about 400 ppm. In general, the polybiguanide compound is present in the composition in an amount greater than about 10 ppm, such as in an amount greater than about 0.001% by weight.

The first anti-microbial agent and the preservative can be present in the wiping composition at various different weight ratios. In one embodiment, the first anti-microbial agent may be present in greater amounts than the second agent or preservative. Alternatively, the preservative may be present in greater amounts than the first anti-microbial agent. For example, the weight ratio between the first anti-microbial agent and the second agent in the wiping composition can generally be from about 50:1 to about 1:10, such as from about 20:1 to about 1:8, such as from about 15:1 to about 1:5, such as from about 10:1 to about 1:3.

In addition to the above components, the wiping composition can also include a liquid carrier. The liquid carrier, for instance, may comprise a polar solvent such as water. The liquid carrier is generally present in the composition in an amount greater than about 20% by weight, such as in an amount greater than about 30% by weight, such as in an amount greater than about 40% by weight, such as in an amount greater than about 50% by weight, such as in an amount greater than about 60% by weight, such as in an amount greater than about 70% by weight, such as in an amount greater than about 80% by weight, such as in an amount greater than about 90% by weight, such as in an amount greater than about 95% by weight, such as in an amount greater than about 96% by weight, such as in an amount greater than about 97% by weight, such as in an amount greater than about 98% by weight, such as in an amount greater than about 99% by weight. In general, the liquid carrier is present in an amount less than about 99.9% by weight.

In addition to a liquid carrier, the wiping composition can also optionally contain one or more organic solvents. The organic solvent, for instance, may comprise an alcohol. Suitable alcohols include, for instance, ethanol, propanol, benzyl alcohol, phenoxyethanol, isopropanol, and mixtures thereof. Alternatively, the organic solvent may comprise a glycol ether. Examples of glycol ethers include diethylene glycol propyl ether, ethylene glycol monomethyl ether, ethylene glycol monoethyl ether, ethylene glycol monopropyl ether, ethylene glycol monoisopropyl ether, ethylene glycol monobutyl ether, diethylene glycol monomethyl ether, diethylene glycol monobutyl ether, diethylene glycol monoethyl ether, diethylene glycol n-butyl ether, ethylene glycol dimethyl ether, ethylene glycol diethyl ether, ethylene glycol dibutyl ether, propylene glycol n-butyl ether, propylene glycol n-propyl ether, tripropylene glycol methyl ether, dipropylene glycol methyl ether, dipropylene glycol butyl ether, dipropylene glycol n-propyl ether, ethylene glycol hexyl ether and combinations thereof. In one embodiment, for instance, the organic solvent present in the composition comprises dipropylene glycol n-propyl ether, ethylene glycol phenyl ether, or mixtures thereof.

When present in the composition, one or more organic solvents can be included in amounts generally greater than about 0.1% by weight, such as in amounts greater than about 0.2% by weight. One or more organic solvents are generally present in the amount less than about 25% by weight, such as in an amount less than about 20% by weight, such as in an amount less than about 15% by weight. In one embodiment, a relatively minor amount of an organic solvent is present in the wiping composition. Although present in a relatively minor amount, however, it is believed that the solvent, in some embodiments, is capable of improving the effectiveness of the composition against various microorganisms, including viruses. For instance, in one embodiment, the organic solvent is present in an amount from about 0.01% by weight to about 2% by weight, such as from about 0.05% by weight to about 0.8% by weight.

In one embodiment, the wiping composition may also contain a pH builder. For example, a pH builder may be added to the composition in order to increase the pH. In one embodiment, the pH builder may comprise an alkanolamine. Particular alkanolamines that may be used as the pH builder include monoethanolamine, 1-Amino-2-Propanol, 3-Amino-1-Propanol, 2-(methylamino)ethanol, 2-(ethylamino)ethanol, 2(propylamino)ethanol, 2(isopropylamino)ethanol, diethanolamine, triethanolamine, diisopropanolamine, triisopropanolamine, or mixtures thereof. Other examples of pH builders include hydroxides such as alkali and alkaline earth metal hydroxides, carbonates, silicates, bicarbonates, and mixtures thereof. For example, the pH builder may comprise sodium metasilicate, such as sodium metasilicate pentahydrate. In one embodiment, more than one pH builder can be present in the wiping composition. For instance, the wiping composition can contain both a silicate and an alkanolamine.

When present, one or more pH builders can be included in the composition in an amount greater than about 0.001% by weight, such as in an amount greater than about 0.01%, such as in an amount greater than about 0.02% by weight, such as in an amount greater than about 0.1% by weight, such as in an amount greater than about 0.2% by weight, such as in an amount greater than 0.3% by weight. One or more pH builders can generally be present in an amount less than about 5% by weight, such as in an amount less than 3% by weight, such as in an amount less than about 2% by weight, such as in an amount less than about 1% by weight.

The wiping composition of the present disclosure can generally have a basic pH. For instance, the pH can generally be greater than about 8, such as greater than about 8.5, such as greater than about 9, such as greater than about 9.5, such as greater than about 10, such as greater than about 10.5. The pH of the wiping composition is generally less than about 14, such as less than about 13.5, such as less than about 13.

In one embodiment, the wiping composition of the present disclosure may also contain a surfactant. Any suitable surfactant may be added to the composition including nonionic surfactants and/or cationic surfactants. The surfactant can generally be present in the wiping composition in an amount greater than about 0.05% by weight, such as an amount greater than about 0.08% by weight. Surfactants are generally present in an amount less than about 20% by weight, such as an amount less than about 15% by weight, such as an amount less than about 10% by weight, such as an amount less than about 5% by weight, such as an amount less than about 3% by weight, such as an amount less than about 2% by weight.

In one embodiment, one or more nonionic surfactants may be present in the wiping composition. A nonionic surfactant, in one embodiment, can include compounds produced by the condensation of a hydrophilic alkylene oxide group with an aliphatic or alkyl aromatic hydrophobic compound. Examples of classes of nonionic surfactants are:
Long chain tertiary amine oxides corresponding to the following general formula:

R₁R₂R₃N→O

wherein R₁ contains an alkyl, alkenyl or monohydroxy alkyl radical of from about 8 to about 18 carbon atoms, up to about 10 ethylene oxide moieties, and up to 1 glyceryl moiety, and R₂ and R₃ contain from 1 to about 3 carbon atoms and up to about 1 hydroxy group, e.g., methyl, ethyl, propyl, hydroxyethyl, or hydroxypropyl radicals. Examples of amine oxides suitable for use in this invention include: dimethyldodecylamine oxide, oleyldi(2-hydroxyethyl)amine oxide, dimethyloctylamine oxide, dimethyldecylamine oxide, dimethyltetradecylamine oxide, di(2-hydroxyethyl)tetradecylamine oxide, 3-dodecoxy-2-hydroxypropyldi(3-hydroxypropyl)amine oxide, and dimethylhexadecylamine oxide.

The polyethylene oxide condensates of alkyl phenols, e.g., the condensation products of alkyl phenols having an alkyl group containing from about 6 to 12 carbon atoms in either a straight chain or branched chain configuration, with ethylene oxide, the said ethylene oxide being present in amounts equal to 6 to 60 moles of ethylene oxide per mole of alkyl phenol. The alkyl substituent in such compounds may be derived from polymerized propylene, diisobutylene, octane, or nonane.

Those derived from the condensation of ethylene oxide with the product resulting from the reaction of propylene oxide and ethylene diamine products which may be varied in composition depending upon the balance between the hydrophobic and hydrophilic elements which is desired. For example, compounds containing from about 40% to about 80% polyoxyethylene by weight and having a molecular weight of from about 5,000 to about 11,000 resulting from the reaction of ethylene oxide groups with a hydrophobic base constituted of the reaction product of ethylene diamine and excess propylene oxide, said base having a molecular weight of the order of 2,500 to 3,000, are satisfactory.

The condensation product of straight or branched chain aliphatic alcohols having from 8 to 18 carbon atoms with ethylene oxide, e.g., a coconut alcohol ethylene oxide condensate having from 10 to 30 moles of ethylene oxide per mole of coconut alcohol, the coconut alcohol fraction having from 10 to 14 carbon atoms.

Particular surfactants that may be used in the composition are nonyl phenol ethoxylates (6-12 moles), primary alcohol ethoxylates (3-12 moles), and secondary alcohol ethoxylates (3-12 moles).

The nonionic surfactants described herein are selected to adsorb or otherwise bond to the fibers of a dry substrate of the cleaning wipe, thereby preventing the active disinfectants from being adsorbed by the fibers of the dry substrate. Without being bound by theory, it is believed that the nonionic surfactants described herein alter the relative equilibrium at the cleaning wipe surface by both modifying the surface to make it less hydrophobic and modifying the wiping composition to make it less hydrophilic. For example, in the case of a quat solution, such as a dialkyl or alkyl benzyl quat solution, the net result is a reduced attraction of the hydrophobic wiper surface for the hydrophobic hydrocarbon tails of the quat solution. The hydrophobic groups have an affinity for the fiber surface of the substrate. Unlike anionic surfactants, the nonionic nature of the surfactant does not attract the cationic quat-based or chlorine-based disinfectant solutions and prevents the active disinfectant from bonding to the substrate fibers.

Exemplary commercially available nonionic surfactants are C₁₁-C₁₅ secondary alkanols condensed with 7, 9, 12, 20, or 40 moles of ethylene oxide (alkyloxypolyethylene oxyethanols), produced by Dow Chemical under the tradenames Tergitol® 15-S-7, 15-S-9, 15-S-12, 15-S-20, and 15-S-40. Additional exemplary nonionic surfactants, of the same type, are marketed by Dow Chemical under the tradenames Tergitol® TMN-6 and TMN-10, believed to comprise reaction products of trimethyl-nonanol with ethylene oxide. Other exemplary nonionic surfactants are commercially available from BASF under the tradename Irgasurf® HL 560. Still other nonionic surfactants include block copolymers of polyoxyethylene and polyoxypropylene that are available under the trade name Pluronic®, marketed by BASF. A single member of any of the foregoing nonionic surfactant compositions can be used in the wiping composition, or mixtures of such exemplary nonionic surfactant materials can be employed.

Another nonionic surfactant that may be present in the wiping composition is a glycoside. For instance, the glycoside may comprise D-glucopyranose, oligomeric, decyl octyl glycosides.

The composition may also contain a chelating agent. Suitable chelating agents include, but are not limited to, ethylene diamine tetra acetic acid (EDTA) and salts thereof (e.g., tetrasodium EDTA available as Versene 100® from Dow Europe S.A. of Horgen, Switzerland), diammonium ethylene diamine tetraacetate, aminocarboxylic acids, amino phosphonic acids, fatty acid salts, mixtures thereof, and the like. Amino phosphonic acids include ethylene diaminetetramethylene phosphonic acid, diethylene triamine penta (methylene phosphonic acid), ethylene diamine tri (methylene phosphonic acid), and hexamethylene diaminetetra(methylene phosphonic acid). In general, preferred chelating agents include methylglycinediacetic acid (MGDA), glutamic acid. N,N-diacetic acid (GLDA), iminodisuccinic acid (IDS); ethylenediaminetetreacetic acid (EDTA) diethylenetriaminepentaacetic acid (DTPA), diethylenethamine-penta-methylene phosphonic acid (DETPMP), hydroxyethyliminodiacetic acid (HEIDA), Nitrilothacetic acid (NTA), aspartic acid diethoxysuccinic acid (AES), aspartic acid-N,N-diacetic acid (ASDA), diethylenethaminepentamethylene-phosphonic acid (DTPMPA), hydroxyethylenediaminetetraacetic acid (HEDTA), hydroxyethylethylenediaminetriacetic acid (HEEDTA), iminodifumaric (IDF), iminoditartahc acid (IDT), iminodimaleic acid (IDMAL), iminodimalic acid (IDM), ethylenediaminedifumaric acid (EDDF), ethylenediaminedimalic acid (EDDM), ethylenediamineditartahc acid (EDDT), ethylenedlaminedimaleic acid and (EDDMAL), aminotri(methylenephosphonic acid) (ATMP). More preferably the chelating agent is selected from iminodisuccinic acid (IDS), ethylenediaminetetraacetic acid (EDTA) diethylenetriaminepentaacetic acid (DTPA), hydroxyethylenediaminetetraacetic acid (HEDTA), hydroxyethylethylenediaminetriacetic acid (HEEDTA), iminodifumaric (IDF), iminoditartaric acid (IDT), iminodimaleic acid (IDMAL), iminodimalic acid (IDM), ethylenediaminedifumaric acid (EDDF), ethylenediaminedimalic acid (EDDM), ethylenediamineditartaric acid (EDDT), ethylenediaminedimaleic acid (EDDMAL) and aminotri(methylenephosphonic acid) (ATMP).

The ability of the chelating agent to remove metal ions facilitates of the solution by preventing hardness (calcium) precipitation. The chelating agent may also serve to bind other metal ions that may adversely affect the effectiveness of the disinfecting components in the composition.

The chelating agent is preferably present in the composition in an amount of from about 0.01% to about 10%, such as from about 0.02% to about 1% based on weight of the composition.

The wiping composition of the present disclosure may optionally further contain corrosion inhibitors, complexing agents, auxiliaries, preservatives, fragrances, colorants and the like. Exemplary corrosion inhibitors include, for example, organic phosphorous compounds and blend of organic phosphorous compounds with a polymeric component. Exemplary auxiliaries include, for example, polyethylene glycol or other similar compounds. Colorants and fragrances may be added provided they do not interfere with the function of the composition and may serve for identifying the composition. Generally, the optional further ingredients will make up less than about 20% by weight of the composition. For example, each of the above identified ingredients or components can be present in the composition generally in an amount from about 0.01% by weight to about 5% by weight. For instance, each of the above components can be present in the composition in an amount from about 0.1% to about 2% by weight, such as from about 0.3% to about 1% by weight.

The wiping composition of the present disclosure can be supplied as a concentrate, which can be diluted prior to use. Alternatively, the disinfecting composition can be provided as a ready-to-use composition.

In one embodiment, when formulated as a concentrate, the wiping composition can contain the first anti-microbial agent which can comprise a quaternary ammonium cation in an amount generally from about 1% to about 50% by weight, such as in an amount from about 1% to about 40% by weight, such as in an amount from about 1% to about 25% by weight. The preservative which may comprise a biguanide compound, on the other hand, can be present in the concentrate in an amount from about 0.5% to about 10% by weight, such as in an amount from about 0.5% to about 5% by weight, such as in an amount from about 0.5% to about 3% by weight. In one embodiment, the concentrate can contain one or more pH builders. The pH builders can be present in an amount from about 0.5% to about 40% by weight. In one embodiment, for instance, a silicate can be present in an amount from about 0.5% to about 5% by weight. Alternatively, an alkanolamine alone or In combination with a silicate can be present in the concentrate in an amount from about 5% to about 40% by weight, such as in an amount from about 10% to about 35% by weight, such as in an amount from about 15% to about 30% by weight.

The concentrate formulated in accordance with the present disclosure can also contain one or more surfactants. For instance, one or more surfactants can be present in the concentrate in an amount from about 2% to about 15% by weight, such as in an amount from about 2% to about 10% by weight. In addition, one or more chelating agents can be present in an amount from about 1% to about 10% by weight, such as in an amount from about 1% to about 8% by weight, such as in an amount from about 1% to about 4% by weight. Optionally, the concentrate can also contain an organic solvent in an amount from about 5% to about 40% by weight, such as in an amount from about 10% to about 35% by weight, such as in an amount from about 15% to about 25% by weight. The remainder of the concentrate can comprise water. Water can generally be present in the concentrate in an amount from about 10% to about 80% by weight, such as in an amount from about 30% to about 70% by weight.

In one embodiment, a concentrate made in accordance with the present disclosure can be deleted with water prior to being impregnated into a wiping substrate. For example, the concentrate can be formulated such that one part concentrate can be combined with from about 100 parts to about 300 parts water. For example, in one embodiment, one part concentrate can be combined with 128 parts water. In an alternative embodiment, the concentrate can be formulated such that 1 part concentrate can be diluted with 256 parts water.

Various different microorganisms may be killed or controlled in accordance with the present disclosure. For instance, the wiping composition of the present disclosure can control gram positive bacteria, gram negative bacteria, and the like. In addition to bacteria, the anti-microbial composition of the present disclosure can also kill and control the growth of various other microorganisms, such as fungi, spores, yeast, mycobacteria, and the like. Of particular advantage, the wiping composition of the present disclosure is particularly well suited for destroying viruses, even non-enveloped viruses such as the Norovirus.

When used as a hard surface cleaner, the wiping composition can be delivered to a surface to be cleaned, sanitized or disinfected by conventional means such as pouring the composition on a surface; a spray; which is applied to a surface via a spray means, including but not limited to, pump spray applicators, pressurized spray applicators and the like; a saturated wipe; a rag and a bucket; a mop and bucket; a sponge and a bucket; or via automated cleaning equipment and other similar and conventional ways to apply an anti-microbial or disinfectant composition to a surface for the purposes of sanitizing or disinfecting the surface.

To use the wiping composition of the present disclosure, a surface is treated with the substrate by spraying, pouring, wiping or otherwise applying the wiping composition to the surface. Once applied to the surface, the wiping composition is allowed to remain on the surface for a period of time. The wiping composition may be applied to the surface and allowed to dry.

Surfaces, which may be disinfected with the compositions include, but are not limited to, those located in dairies, homes, health care facilities, canneries, food processing plants, restaurants, hospitals, institutions, and industry. Any suitable hard surface may be treated in accordance with the present disclosure, particularly frequently touched hard surfaces. The hard surface, for instance, can be made from glass, a metal such as an aluminum or stainless steel, a ceramic, a stone such as granite or marble, a plastic or polymer material, or the like. Specific areas targeted for application include hard surfaces in the home such as kitchen countertops, cabinets, appliances, waste cans, laundry areas, garbage pails, bathroom fixtures, toilets, water tanks, faucets, mirrors, vanities, tubs, and showers. The compositions can also be used to sanitize floors, walls, furniture, mirrors, toilet fixtures, windows, and wood surfaces, such as fence rails, porch rails, decks, roofing, siding, window frames, and door frames. Areas in hospitals would include beds, gurneys, tables, canisters, toilets, waste cans, stands, cabinets, shower stalls, floors, door knobs, bed rails, walls or any other non-porous surface.

One particularly useful application method is to impregnate the wiping composition into a wipe substrate. In this embodiment, the wipe is a single use wipe that is impregnated with the wiping composition and is stored in a container that will dispense the wipe to a user. The container with the wipes may contain a single wipe, or several wipes. Suitable containers include a pouch containing a single wipe, such as a moist towelette which is torn open by the user, or may be a pouch with a resealable opening containing several wipes in a stacked fashion, a rolled fashion or other suitable formation that would allow a single wipe to be removed from the opening at a time. Pouches are generally prepared form a fluid impervious material, such as a film, a coated paper or foil or other similar fluid impervious materials. In another way to dispense wipes of the present disclosure is to place the wipe in to a fluid impervious container having an opening to access the wipes in the container. Containers may be molded plastic container with lids that are fluid impervious. Generally, the lid will have an opening to access the wipes in the container. The wipe in the container may be in an interleaved stack, such that as a wipe is removed from the container, the next wipe is positioned in the opening of the container ready for the user to remove the next wipe. Alternatively, the wipe may be a continuous material which is perforated between the individual wipes of the continuous material. The continuous wipe material with perforations may be in a folded form or may be in a rolled form. Usually, in the rolled form, the wipe material is fed from the center of the rolled material. As with the interleaved stack, as a wipe is removed from the container, the next wipe is positioned in the opening to facilitate removal of the next wipe, when needed.

Disposable wipes provide advantages over other application vehicles, such as a reusable sponge, rag or the like. Unlike sponges, rags and the like, which are used repeatedly, the impregnated wipe is used a single time and disposed of. Reused sponges or rags present problems since the sponges or rags may carry microbes that are not easily killed by the disinfecting composition.

The wiping composition can be impregnated into the wipe such that the wipe is pre-moistened and will express or release the wiping composition onto the surface as the wipe is run across the surface to be treated. Generally, the wiping composition is saturated into the wipe such that the wipe will release the wiping composition to the surface through the wiping action. Generally, the wiping composition is used from about 2 parts to 10 parts by weight per 1 part by weight of the wiper substrate, more preferably from about 3 to about 5 parts by weight per 1 part by weight of the wiper substrate. In these ranges, complete saturation of the substrates can be achieved. It is noted that the amount of the wiping solution may go up or down to achieve complete saturation of the wipe substrate, depending on the particular wipe substrate.

Suitable wipe substrates include woven and nonwoven materials. Essentially any nonwoven web material may be used. Exemplary nonwoven materials may include, but are not limited to meltblown, coform, spunbond, airlaid, hydroentangled nonwovens, spunlace, bonded carded webs, and laminates thereof. Optionally, the nonwoven may be laminated with a film material as well. The fibers used to prepare the wipe substrate may be cellulosic fiber, thermoplastic fibers and mixtures thereof. The fibers may also be continuous fibers, discontinuous fibers, staple fibers and mixtures thereof. Basis weights of the nonwoven web may vary from about 12 grams per square meter to 200 grams per square meter or more.

In one embodiment the wipe is impregnated with a liquid component containing both active and inert ingredients within the allowable tolerance levels and the wiping composition expressed from the wipe contains active ingredients within the allowable tolerance levels. Once applied to the surface, the anti-microbial wiping composition is allowed to remain on the surface for a period of time.

The following examples are intended to provide a more complete understanding of the present invention. The examples are not intended, however, to limit the invention.

### EXAMPLES

### Example No. 1

Various different wiping compositions were formulated and tested.

In Table 1, the formulas were loaded onto wipe substrates with a 4 part liquid and 1 part wipe (4:1) loading ratio, except for Sample No. 4 which was loaded into the substrate at a 5 part liquid to 1 part wipe loading ratio. The substrate material was a woven material made from 100% polypropylene fibers except Sample No. 4. In Sample No. 4, the substrate material was a 100% polyester material.

| Ingredient | Sample No. 1 | Sample No. 2 | Sample No. 3 | Sample No. 4 |
|---|---|---|---|---|
| Alkyl dimethyl benzyl and | 0.274 | 0.274 | 0.385 | — |
| dialkyl dimethyl ammonium | | | | |
| chloride | | | | |
| Didecyl dimethyl ammonium | — | — | — | 0.034 |
| carbonate/bicarbonate | | | | |
| Sodium metasilicate | 0.026 | 0.026 | 0.026 | — |
| pentahydrate | | | | |
| Monoethanolamine | 0.427 | — | 0.445 | — |
| Tetrasodium EDTA | 0.051 | 0.051 | 0.053 | — |
| C11-C15 alkyl | 0.100 | 0.100 | 0.105 | — |
| poly(ethyleneoxy)ethanol | | | | |
| Poly(hexamethylene | — | 0.045 | 0.050 | 0.045 |
| biguanide hydrochloride) | | | | |
| D-Glucopyranose, oligomeric, | — | — | — | 0.143 |
| decyl octyl glycosides | | | | |
| Isopropanol | — | — | — | 0.409 |
| Water | q.s. to 100 | q.s. to 100 | q.s. to 100 | q.s. to 100 |

The presaturated wiping products were then evaluated for virucidal efficacy. In particular, the presaturated wipes were tested according to the Presaturated Towelette Test which is modified test AOAC 961.02 in accordance with ASTM Test E2362. The presaturated wipes were tested on a hard surface against the virus Feline Calicivirus, which also serves as a surrogate virus for Norovirus.

Feline Calicivirus, also a member of the Caliciviridae family, serves as a valuable model virus for efficacy testing of Norovirus, since these viruses share many similar characteristics and Feline Calicivirus can be propagated in cell cultures. The test procedure was to simulate the way in which the product is intended to be used. This method is in compliance with the requirements of the U.S. Environmental Protection Agency (EPA). According to the regulatory agency, the test samples passes the Pre-Saturated or Impregnated Towelette Virucidal Efficacy Test if there is complete inactivation of the challenge virus at all dilutions. When cytotoxicity is evident, at least a three Log₁₀ reduction in titer must be demonstrated beyond the cytotoxic level. The micro efficacy test results are shown in Table 2.

| | Wipe substrates impregnated with | | | |
|---|---|---|---|---|
| | Sample | Sample | Sample | Sample |
| | No. 1 | No. 2 | No. 3 | No. 4 |
| Contact Time | 9 min. 45 | 9 min. 45 | 10 minutes | 5 minutes |
| | seconds | seconds | | |
| Inactivation of | Incomplete | Complete | Complete | Complete |
| Feline Calicivirus | | | | |
| Log₁₀ Reduction | ≥4.37 | ≥4.47 | 3.07 | ≥4.92 |
| Efficacy results | Fail | Pass | Pass | Pass |

As shown above, the formulations containing a first anti-microbial agent and a second anti-microbial agent were effective against the tested virus while Sample No. 1 failed the test.

### Example No. 2

Various different wiping compositions were formulated and tested similar to the procedures described in Example No. 1. In particular, the following formulations were tested:

| Ingredient | Sample No. 5 | Sample No. 6 |
|---|---|---|
| Dipropylene glycol | 0.150 | 0.150 |
| n-propylether | | |
| Ethylenediaminetetroaacetic | 0.02 | — |
| acid | | |
| Lactic acid | — | 0.150 |
| Monoethanolamine | 0.026 | 0.030 |
| Tetrasodium EDTA | — | 0.0076 |
| Bis(3- | 0.550 | 0.350 |
| aminopropyl)dodecylamine | | |
| Poly(hexamethylene | — | 0.100 |
| biguanide hydrochloride) | | |
| Water | q.s. to 100 | q.s. to 100 |

Presaturated wiping products made with the above formulations were then evaluated for virucidal efficacy. In particular, the presaturated wipes were tested according to the Presaturated Towelette Test which is modified test AOAC 961.02 in accordance with ASTM Test E2362. The presaturated wipes were tested on a hard surface against the virus Feline Calicivirus, which also serves as a surrogate virus for Norovirus.

The micro efficacy test results are shown below.

| | Wipe substrates impregnated | |
|---|---|---|
| | with | |
| | Sample | Sample |
| | No. 5 | No. 6 |
| Contact Time | 3 min. | 3 min. |
| Efficacy results | Fail | Pass |

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1.-11. (canceled)

12. A premoistened wiping product comprising:
a liquid absorbent substrate; and
a wiping composition contained within the substrate, the wiping composition comprising an aqueous solution containing a first anti-microbial agent combined with a polybiguanide or salt thereof, the first anti-microbial agent comprising a quaternary ammonium cation or a tertiary amine.

13. A premoistened wiping product as defined in claim 12, wherein the first anti-microbial agent comprises a halide salt of a quaternary ammonium cation.

14. A premoistened wiping product as defined in claim 12, wherein the first anti-microbial agent comprises an alkyl dimethyl benzyl ammonium chloride, a dialkyl dimethyl ammonium chloride, an alkyl dimethyl ethyl benzyl ammonium chloride, or mixtures thereof.

15. A premoistened wiping product as defined in claim 12, wherein the first anti-microbial agent comprises a carbonate/bicarbonate salt of a quaternary ammonium cation.

16. A premoistened wiping product as defined in claim 12, wherein the first anti-microbial agent is present in relation to the polybiguanide or salt thereof at a weight ratio of from about 50:1 to about 1:10.

17. A premoistened wiping product as defined in claim 12, wherein the wiping composition further contains a nonionic surfactant.

18. A premoistened wiping product as defined in claim 17, wherein the nonionic surfactant comprises a glycoside or a C11-C15 alkanol containing ethylene oxide units.

19. A premoistened wiping product as defined in claim 12, wherein the wiping composition contains water in an amount greater than about 90% by weight.

20. A premoistened wiping product as defined in claim 12, wherein the wiping composition further contains an organic solvent.

21. A premoistened wiping product as defined in claim 12, wherein the wiping composition further contains a chelating agent.

22. A premoistened wiping product as defined in claim 12, wherein the liquid absorbent substrate comprises a nonwoven web.

23. A premoistened wiping product as defined in claim 12, wherein the liquid absorbent substrate comprises a meltblown web, a coform web, a spunbond web, an airlaid web, an airlaced web, a hydroentangled web, a bonded carded web, or a laminate thereof.

24. A premoistened wiping product as defined in claim 12, wherein the first anti-microbial agent comprises the tertiary amine.
